(19) United States
(12) Patent Application Publication
(10) Pub. No.: US 2006/0111638 A1
(43) Pub. Date: May 25, 2006

(54) Noninvasive pulse measurement method and system with constant pressure for extremities of a human body

(75) Inventors: Hen-Hong Chang, Taichung (TW); Kang-Ping Lin, ChungLin (TW); Wen-Chen Lin, MiaoLi (TW)

Correspondence Address: NIKOLAI & MERSEREAU, P.A., 900 SECOND AVENUE SOUTH, SUITE 820, MINNEAPOLIS, MN, 55402, US

(73) Assignee: Hen-Hong Chang, TaiChung (TW)

(21) Appl. No.: 11/126,612

(22) Filed: May 11, 2005

(30) Foreign Application Priority Data
Nov. 24, 2004 (TW) 093136192

(51) Int. Cl. A61B 5/02

(52) U.S. Cl. 600/490

(57) ABSTRACT

The invention is related to a noninvasive pulse measurement system with constant pressure for extremities of a human, and the constant pressure is modulated among 30 to 120 mmHg. There are an auto-mode and a manual mode for measurement algorithm. The auto-mode completes measurement and recording processes automatically during 2 minutes and pressurizes the system from low pressure (30 mmHg) to high pressure (120 mmHg) with 10 mmHg gradient every 5 seconds while the manual mode completes measurement and recording processes during a long time under a user's setting. Moreover, by a post-treatment of the system, the system is able to obtain continuous pulse signals, continuous pulse differential signals, and continuous pulse integral signals that are useful for Chinese medicine to evaluate and monitor the user's cardio-physiology and circulatory system.

### BACKGROUND OF THE INVENTION

1. Field of the Invention

The present invention relates to a noninvasive pulse measurement system for Chinese medicine, which provides a measurement method to determine a patient's pulse pressure parameters responsive to the provision of a constant pressure, so as to not only facilitate the anamnesis establishment of the patient but also provide information for a doctor of traditional Chinese medicine to monitor and analyze the patient's condition for a long time.

2. Description of Related Art

With the advancement of biotechnology, pulse measurement systems have developed from invasive measurement to noninvasive measurement, as shown in FIG. 1, showing a block diagram of a conventional noninvasive pulse measurement system, comprising an analog circuit (10) and a digital circuit (12). The analog circuit (10) comprises a pressure sensor (100), a pre-amplifier (102), a high pass filter (104), a low pass filter (106), and a gain controller (108). The digital circuit (12) comprises an analog-to-digital conversion circuit (120), a microprocessor (122), a graphic liquid crystal display (124), a pressure motor (126) and a decompression valve (128).

In traditional Chinese medicine, a doctor feels a patient's pulse by pressing a finger on the patient's wrist. According to different pressures provided by the doctor, the doctor can describe the responses of the patient's pulse and then makes the diagnosis of the patient's condition. However, the provision of various pressures by the doctor is not easy to evaluate, so there appears the conventional noninvasive pulse measurement system in the market. With the conventional noninvasive pulse measurement system, using one or more pressure sensors vertically set on the patient's arteria radialis to measure the pulse is not convenient and is difficult to operate.

To overcome the shortcomings, the present invention tends to provide a noninvasive pulse measurement system having a convenient cuff [having been] used with a conventional hemadynamometer, and an pulse measurement method to impose a precise constant pressure on the patient's blood vessel and record the responses of the pulse.

### SUMMARY OF THE INVENTION

The primary objective of the present invention is to provide a noninvasive pulse measurement system that is able to continuously monitor pulse signals at different constant pressures to simulate a doctor's feeling of a patient's pulse as practiced in traditional Chinese medicine, and record the responses of the pulse to the pressures so as to provide useful application to diagnosis for Chinese medicine, cardio-physiology and circulatory system.

To attain the objective mentioned above, the present invention provides a noninvasive pulse measurement method and system with constant pressure for extremities, characterized in that the system can provide a constant pressure for a cuff for a long time and continuously measure a person's extremities arterial pulse signals. The pulse measurement method is very easily carried out by programming firmware, which has two measurement modes: an auto mode and a manual mode. In the auto mode the system can quickly obtain the patient's pulse signals by providing ten different constant pressures, and thereafter, in manual mode the patient can arbitrarily adopt a constant pressure to measure his/her pulse signals. Thus, the system is able to record, monitor and analyze the patient's systolic pressure, diastolic pressure and average blood pressure.

Other objects, advantages and novel features of the invention will become more apparent from the following detailed description when taken in conjunction with the accompanying drawings.

### BRIEF DESCRIPTION OF THE DRAWINGS

The objects, spirits and advantages of the preferred embodiments of the present invention will be readily understood by the accompanying drawings and detailed descriptions, wherein:

FIG. 1 is a block diagram of the conventional noninvasive pulse measurement system;

FIG. 2 is a perspective view of the noninvasive pulse measurement system in accordance with the present invention;

FIG. 3 is a block diagram of the noninvasive pulse measurement system of FIG. 2;

FIG. 4A is an auto mode flow chart of the noninvasive pulse measurement method in accordance with the present invention; and

FIG. 4B is a manual mode flow chart of the noninvasive pulse measurement method in accordance with the present invention.

### DETAILED DESCRIPTION OF THE PREFFERED EMBODIMENT

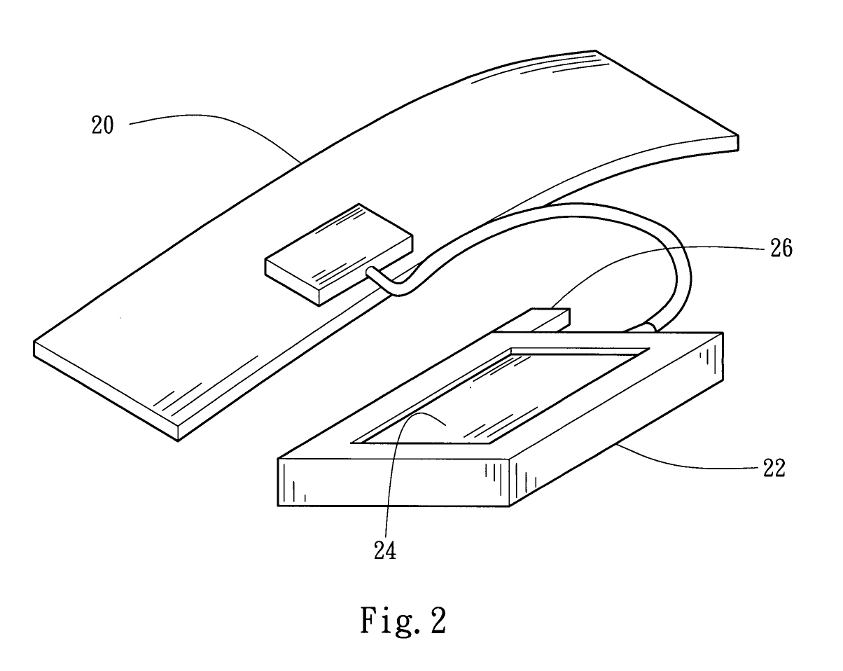

FIG. 2 is a perspective view of the noninvasive pulse measurement system in accordance with the present invention. The noninvasive pulse measurement system comprises: a cuff (20), a main circuit body (22), display device (24), and a storage device (26), wherein the display device (24) is usually a graphic crystal display device to display instant pulse signals, pulse differential signals, pulse integral signals, heartbeat frequency, and pressure in the cuff. The storage device (26) is an extensible memory card, such as Compact Flash card or Smart Media card.

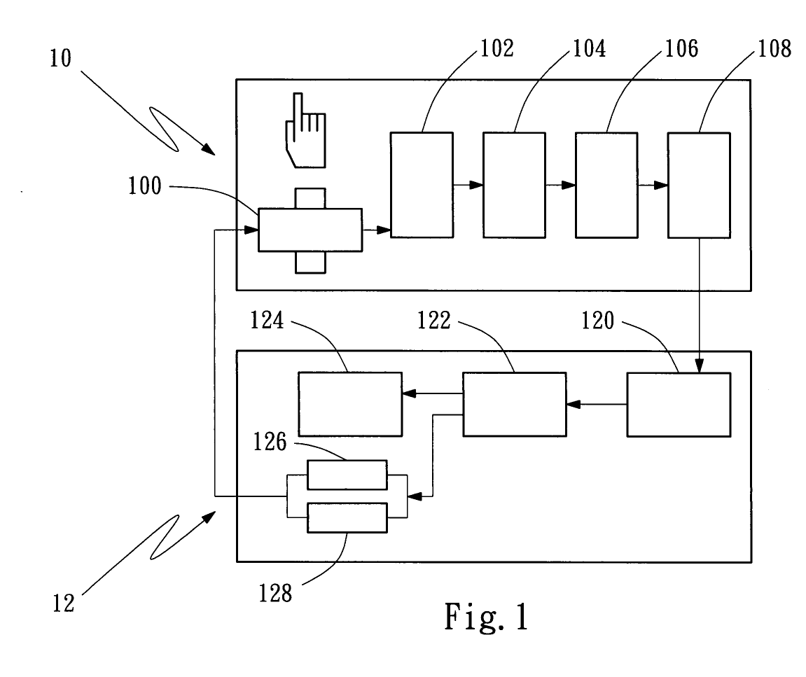

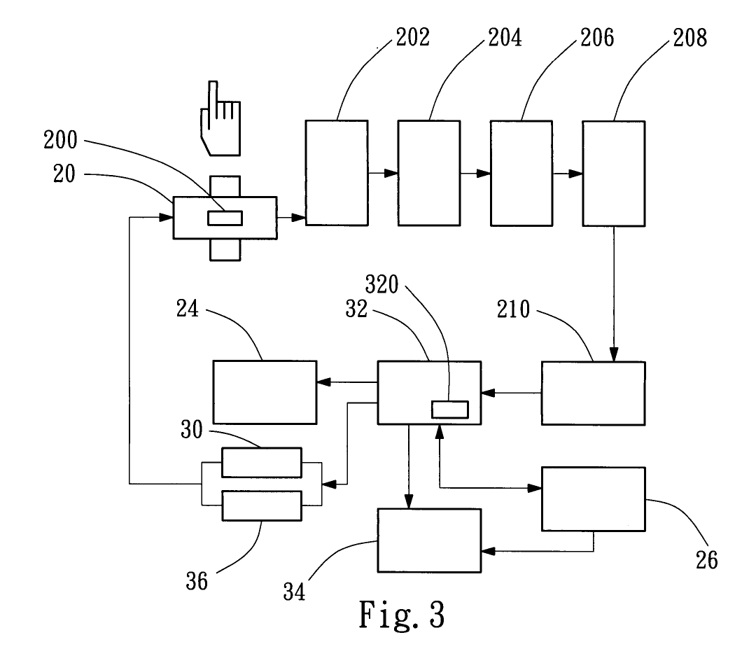

FIG. 3 is a block diagram of the noninvasive pulse measurement system of FIG. 2. In addition to the elements similar to those of the conventional noninvasive pulse measurement system as shown in FIG. 1, that is, a pressure sensor (200), a pre-amplifier (202), a high pass filter (204), a low pass filter (206), a gain controller (208), an analog-to-digital conversion circuit (210), a microprocessor (32), a graphic liquid crystal display (24), a pressure motor (30) and a decompression valve (36) etc., the noninvasive pulse measurement system of the present invention further comprises the storage device (26) and a return mechanism, in which the storage device (26) is electrically connected to the microprocessor (32) to store data processed by the microprocessor (32) and also can transmit the stored data therein to a personal computer (34) with a standard computer transmission interface to form the return mechanism. Furthermore, the microprocessor (32) has a two-way COM port (320) to transmit the processed data to the personal computer (34) to be stored, dealt with, or analyzed. In this embodiment of the present invention, the two-way COM port (320) is the type of RS-232 interface.

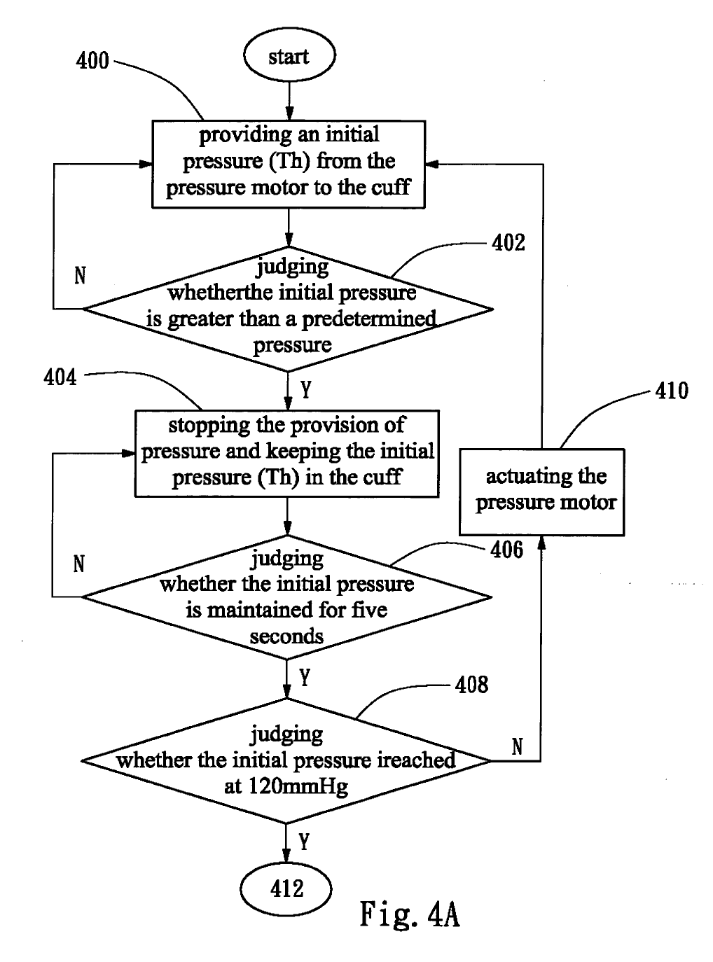

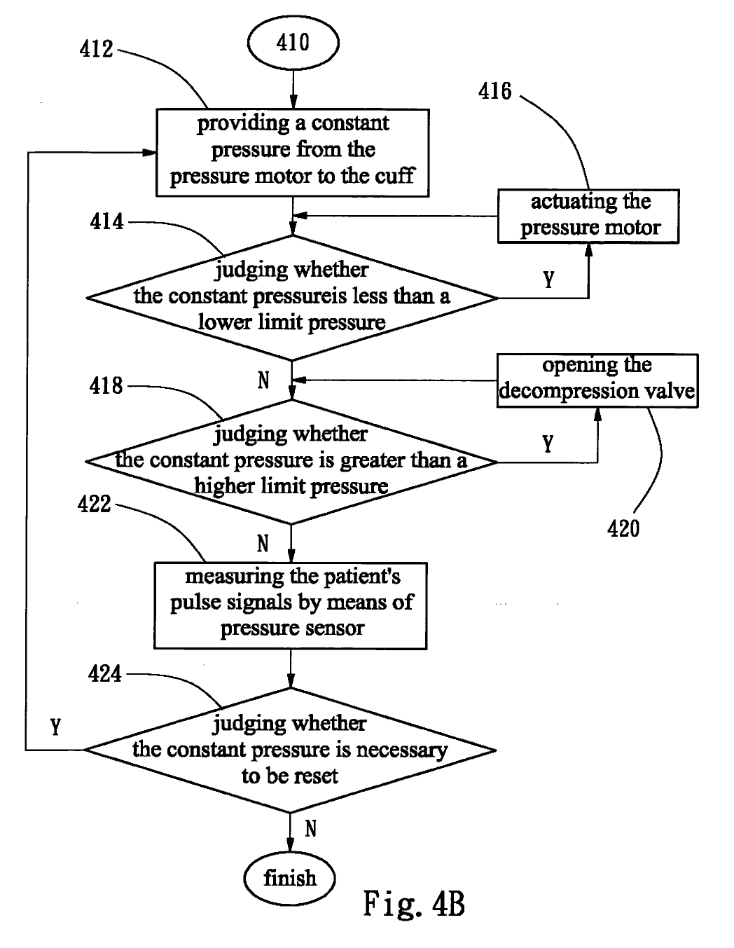

In the aspect of the firmware, the microprocessor (32) at least comprises a code of continuous pulse measurement. The pulse measurement flow path is shown in FIGS. 4A and 4B. In the case of the code processed by the microprocessor (32), the pulse measurement system executes the following steps: first, providing an initial pressure (Th) from the pressure motor (30) to the cuff (20), step (400); then, judging whether the initial pressure (Th) is greater than a predetermined pressure (Po), step (402), which is 30 mmHg in this embodiment; if greater, stopping the provision of pressure and keeping the initial pressure (Th) in the cuff (20), step (404); if not greater, returning to step (400); next, measuring a patient's pulse signal by means of the pressure sensor (200) responsive to the initial pressure (Th) and transmitting the signal to the microprocessor (32) with the conversion by the analog-to-digital conversion circuit (210) to calculate the patient's physiological function parameters, and meanwhile, judging whether the initial pressure (Th) is maintained for a predetermined period, step (406), which is five seconds in this embodiment; if not maintained so, returning to step (404); if maintained so, judging whether the initial pressure (Th) is reached at a maximum pressure (Pmax), step (408), which is 120 mmHg in this embodiment; if not reached, actuating the pressure motor (30) to provide a rising pressure (Pup), step (410), which is 10 mmHg in this embodiment, added on the predetermined pressure (Po), and then stopping operation of the pressure motor (30) and returning to step (400); if reached, opening the decompression valve (36) to decompress the cuff (20) until 0 mmHg is achieved. The aforementioned steps all belong to the auto mode of the pulse measurement method to measure the patient's responsive pulse signals corresponding to the different pressures, such that the pulse signals can be referred as a reference index for the manual mode measurement of the pulse measurement method.

Thereafter, the pulse measurement system executes the manual mode, which comprises, the following steps, as shown in FIG. 4B: providing a constant pressure (Ps) from the pressure motor (30) to the cuff (20), step (412); judging whether the constant pressure (Ps) is less than a lower limit pressure (Pslow), step (414); if less, actuating the pressure motor (30) to provide a pressure, step (416), and then, stopping the action of the pressure motor (30) and returning to step (414); if not less, judging where the constant pressure (Ps) is greater than a higher limit pressure (Pshigh), step (418); if greater, opening the decompression valve (36) to decompress the cuff (20), step (420) and then returning to step (418); if not greater, measuring the patient's pulse signals by means of pressure sensor (200) to calculate the pulse differential signal and pulse integral signal; finally, judging whether the constant pressure (Ps) is necessary to be reset, step (424); if necessary, returning to step (412); if not necessary, finishing the measurement method. Of course, after finishing the measurement method, the manual mode can automatically return to step (414).

Accordingly, step (414) and step (418) ensure that the pressure in the cuff (20) is maintained constant permanently, in which the lower limit pressure (Pslow) is lower than the constant pressure (Ps) while the higher limit pressure (Pshigh) is higher than the constant pressure (Ps), and the value of the constant pressure (Ps) is by manual setting, the range of which is of 5 to 180 mmHg.

Therefore, the features of the present invention features are as follows:

The pulse measurement method is easily carried out by programming firmware, which has two measurement modes: an auto mode and a manual mode. In the auto mode the system can complete the measurement and signal recording automatically, while in manual mode the patient can arbitrarily adopt a constant pressure to measure his or her pulse signals to monitor and record the his or her physiological condition.

The pulse measurement system in combination with the extensible storage device, such as Compact Flash card, or Smart Media card, is able to store a large quantity of data of the pulse signals for a long time.

The pulse measurement system is able to transmit the pulse signals to a remote computer by means of the standard computer transmission interface so as to allow follow-on analysis and treatment.

Although this invention has been disclosed and illustrated with reference to particular embodiments, the principles involved are susceptible for use in numerous other embodiments that will be apparent to persons skilled in the art. This invention is, therefore, to be limited only as indicated by the scope of the appended claims.

What is claimed is:

1. A noninvasive pulse measurement method with constant pressure for extremities of a human body, comprising the steps of:
(a) providing an initial pressure (Th) from a pressure motor to a cuff,
(b) judging whether the initial pressure (Th) is greater than a predetermined pressure (Po), if the initial pressure is greater, stopping the provision of pressure and keeping the initial pressure (Th) in the cuff for a predetermined period of time;
(c) measuring a user's pulse signal by means of a pressure sensor responsive to the initial pressure (Th) and calculating the user's physiological function parameters;
(d) judging whether the initial pressure (Th) is maintained for the predetermined period of time, if the initial pressure is maintained, going to the next step;
(e) judging whether the initial pressure (Th) reaches a maximum pressure (Pmax), if the maximum pressure is reached, opening the decompression valve to decompress the cuff, and going to the next step;
(f) providing a constant pressure (Ps) from the pressure motor to the cuff;
(g) judging whether the constant pressure (Ps) is less than a lower limit pressure (Pslow), if the pressure is not less, going to the next step;
(h) judging whether the constant pressure (Ps) is greater than a higher limit pressure (Pshigh), if the constant pressure is not greater, going to the next step;
(i) measuring the user's pulse signal by means of the pressure sensor responsive to the constant pressure (Ps) and calculating the user's physiological function parameters;
(j) judging whether the constant pressure (Ps) needs to be reset, if not necessary, finishing the measurement method.

2. The noninvasive pulse measurement method of claim 1, wherein if the pressure is not greater in the step (b), returning to the step (a).

3. The noninvasive pulse measurement method of claim 1, wherein if the initial pressure is not maintained in the step (d), returning to the step (c).

4. The noninvasive pulse measurement method of claim 1, wherein if the maximum pressure is not reached in the step (e), adding a rising pressure (Pup) to the cuff and returning to the step (a).

5. The noninvasive pulse measurement method of claim 1, wherein if the pressure is less in the step (g), actuating the pressure motor to provide a pressure.

6. The noninvasive pulse measurement method of claim 1, wherein if the constant pressure is greater in the step (h), opening the decompression valve to decompress the cuff.

7. The noninvasive pulse measurement method of claim 1, wherein if the constant pressure needs to be reset, in the step (0), returning to the step (f).

8. A noninvasive pulse measurement system with constant pressure for extremities of a human body, the system comprising a cuff, a pressure sensor, a pre-amplifier, a high pass filter, a low pass filter, a gain controller, a pressure motor, a decompression valve, an analog-to-digital conversion circuit, and a display device, characterized in that:
the noninvasive pulse measurement system further comprises a storage device connected to the microprocessor to store the data processed by the microprocessor;
the microprocessor further comprises a two-way COM port to transmit the data processed by the microprocessor to a computer; and
the microprocessor further comprises a code of continuously pulse measurement to execute a measurement method comprising following steps:
(1) providing an initial pressure (Th) from the pressure motor to the cuff;
(2) judging whether the initial pressure (Th) is greater than a predetermined pressure (Po), if the initial pressure is greater, stopping the provision of pressure and keeping the initial pressure (Th) in the cuff for a predetermined period of time;
(3) measuring a user's pulse signal by means of the pressure sensor responsive to the initial pressure (Th) and calculating the user's physiological function parameters;
(4) judging whether the initial pressure (Th) is maintained for the predetermined period of time, if the initial pressure is maintained, going to the next step;
(5) judging whether the initial pressure (Th) reaches a maximum pressure (Pmax), if the initial pressure reaches the maximum, opening the decompression valve to decompress the cuff, and going to the next step;
(6) providing a constant pressure (Ps) from the pressure motor to the cuff;
(7) judging whether the constant pressure (Ps) is less than a lower limit pressure (Pslow), if the constant pressure is not less, going to the next step;
(8) judging where the constant pressure (Ps) is greater than a higher limit pressure (Pshigh), if the constant pressure is not greater than the higher limit pressure, going to the next step;
(9) measuring the user's pulse signal by means of the pressure sensor responsive to the constant pressure (Ps) and calculating the user's physiological function parameters;
(10) judging whether the constant pressure (Ps) needs to be reset, if not necessary to reset the constant pressure, finishing the measurement method.

9. The noninvasive pulse measurement system of claim 8, wherein the two-way COM port is a type of RS-232 interface.

10. The noninvasive pulse measurement system of claim 8, wherein the storage device is an extensible memory card selected from the group consisting of a Compact Flash card, or a Smart Media card.

11. The noninvasive pulse measurement system of claim 8, wherein the display device is a liquid crystal display device.

12. The noninvasive pulse measurement system of claim 8, wherein if the initial pressure is not greater in the step (2), returning to the step (1).

13. The noninvasive pulse measurement system of claim 8, wherein if the initial pressure is not maintained in the step (4), returning to the step (3).

14. The noninvasive pulse measurement system of claim 8, wherein if the initial pressure does not reach the maximum in the step (5), adding a rising pressure (Pup) to the cuff and returning to the step (1).

15. The noninvasive pulse measurement system of claim 8, wherein if the constant pressure is less than the lower limit in the step (7), actuating the pressure motor to provide a pressure.

16. The noninvasive pulse measurement system of claim 8, wherein if the constant pressure is greater than the higher limit in the step (8), opening the decompression valve to decompress the cuff.

17. The noninvasive pulse measurement system of claim 8, wherein if it is necessary to reset the constant pressure in the step (10), returning to the step (6).
